(19) United States
(12) Patent Application Publication
(10) Pub. No.: US 2026/0216445 A1
(43) Pub. Date: Jul. 30, 2026

(54) Medicament delivery device

(71) Applicant: SHL Medical AG, Zug (CH)

(72) Inventors: Amit Singh, Deerfield Beach, FL (US); Nurettin Ali, Deerfield Beach, FL (US)

(21) Appl. No.: 19/143,035

(22) PCT Filed: Dec. 21, 2023
(86) PCT No.: PCT/EP2023/087173

§ 371 Date: Jun. 25, 2025

Related U.S. Application Data
(60) Provisional application No. 63/436,981, filed on Jan. 4, 2023.

(30) Foreign Application Priority Data
Feb. 2, 2023 (EP) 23154580.7

(51) Int. Cl. A61M 5/32

(52) U.S. Cl. CPC A61M 5/3202

(57) ABSTRACT

The present disclosure relates to a medicament delivery device, the medicament delivery device having a proximal end pointing towards a dose delivery site during use of the medicament delivery device, and a distal end opposite the proximal end. The medicament delivery device includes a housing comprising a longitudinal axis, a medicament delivery member cover axially moveable within the housing between an extended position and a retracted position, and a cap. The cap comprises an outer housing and an inner housing concentrically provided radially inside the outer housing with respect to the longitudinal axis. A space between the outer housing and the inner housing is configured to receive a proximal end of the medicament delivery member cover extending towards a medicament delivery site. The medicament delivery member cover comprises a flexible medicament delivery member cover lock being flexible in the radial direction. The medicament delivery member cover lock comprises an outer protrusion extending in the radial outward direction and being configured to be received in a corresponding housing slot. The inner housing is configured to block inward radial movement of the medicament delivery member cover lock.

### TECHNICAL FIELD

The present disclosure relates to a medicament delivery device and in particular to a lock mechanism for a medicament delivery device before use.

### BACKGROUND

A medicament delivery device, in particular an autoinjector, according to the prior art comprises a housing comprising a front housing and a rear housing. The housing is adapted to hold a medicament container, such as a syringe. The syringe may be a pre-filled syringe and have a needle as a medicament delivery member arranged at a proximal end. When the autoinjector and/or the syringe are assembled, a protective needle sheath may be removably coupled to the needle. The protective needle sheath may be made of a flexible shell (for instance composed of rubber) and/or a rigid shell (for instance composed of plastic). A stopper, also called plunger, may be arranged for sealing the syringe distally and for expelling a medicament contained in the syringe through the needle. In other exemplary embodiments, the medicament container may be a cartridge which includes the medicament and engages a removable needle (e.g., by threads, snaps, friction, etc.).

A cap may be removably disposed at a proximal end of the housing. The cap may include an element (e.g., a barb, a hook, a narrowed section, etc.) arranged to engage the protective needle sheath, the housing and/or a medicament delivery member cover, also referred to as needle cover, telescoped within the housing. The cap may comprise grip features for facilitating removal of the cap (e.g., by twisting and/or pulling the cap relative to the housing).

A medicament delivery member cover biasing member (for instance a coil spring) may be arranged to bias the medicament delivery member cover in a proximal direction relative to the housing, before injection, for safety reasons. The medicament delivery member cover biasing member needs to be compressed at the beginning of injection in order to retract the medicament delivery member cover inside the housing and uncover the medicament delivery member, thereby activating the device.

A pre-loaded drive biasing member, for instance a pre-compressed drive coil spring, may be arranged within the housing. A plunger rod may be used to forward force from the drive biasing member to the stopper. In an exemplary embodiment, the plunger rod is hollow and the drive biasing member is arranged within the plunger rod. In another exemplary embodiment, the plunger rod is solid and the drive biasing member engages a distal end of the plunger rod. When the pre-loaded drive biasing member is released, the drive biasing member biases the plunger rod in the proximal direction relative to the housing, releasing the plunger rod. Besides that, the plunger rod may itself comprise means for sealing the medicament container such that no stopper needs to be provided.

A plunger rod release mechanism may be arranged for preventing release of the plunger rod prior to retraction of the medicament delivery member cover relative to the housing and for releasing the plunger rod only once the medicament delivery member cover is sufficiently retracted.

A medicament delivery member cover lock mechanism (needle cover lock mechanism) may be arranged to prevent retraction of the medicament delivery member cover relative to the housing when the cap is in place, thereby avoiding unintentional activation of the autoinjector (e.g., if dropped, during shipping or packaging, etc.).

However, known mechanisms may be overly complicated to manufacture and subject to fatigue such that reliability of the needle cover lock is not always ensured.

Thus, there is a need to improve safety of medicament delivery devices and in particular the locking mechanisms of the needle cover prior to medicament delivery.

This need is met by the present disclosure.

### SUMMARY

Reference should now be made to the appended claims.

According to a first aspect, the invention relates to a medicament delivery device, the medicament delivery device having a proximal end pointing towards a dose delivery site during use of the medicament delivery device, and a distal end opposite the proximal end, the medicament delivery device comprising:
a housing comprising a longitudinal axis,
a medicament delivery member cover axially moveable within the housing between an extended position and a retracted position,
a cap,
wherein the cap comprises an outer housing and an inner housing concentrically provided radially inside the outer housing with respect to the longitudinal axis,
wherein a space between the outer housing and the inner housing is configured to receive a proximal end of the medicament delivery member cover extending towards a medicament delivery site,
wherein the medicament delivery member cover comprises a flexible medicament delivery member cover lock being flexible in the radial direction,
wherein the medicament delivery member cover lock comprises an outer protrusion extending in the radial outward direction and being configured to be received in a corresponding housing slot, and
wherein the inner housing is configured to block inward radial movement of the medicament delivery member cover lock.

In a non-limiting embodiment, the medicament delivery member cover lock comprises an inner protrusion extending in the radial inward direction.

In a non-limiting embodiment, the outer protrusion is configured to abut a distal end of the housing slot when the medicament delivery member cover is moved in the distal direction, and the abutment blocks further distal movement of the medicament delivery member cover.

In a non-limiting embodiment, the distal end of the housing slot is sloped radially inwards and configured to bias the medicament delivery member cover lock radially inwards when the medicament delivery member cover is moved in the distal direction.

In a non-limiting embodiment, when the cap is attached to the housing and the medicament delivery member cover is moved in the distal direction, the medicament delivery member cover lock is biased radially inwards and the inner protrusion is configured to abut the inner housing thus blocking radial movement of the medicament delivery member cover lock, and when the cap is removed from the housing and the medicament delivery member cover is moved in the distal direction, the medicament delivery member cover lock is biased radially inwards and the outer protrusion is disengaged from the housing slot thus allowing distal movement of the medicament delivery member cover.

According to a second aspect, the invention relates to a medicament delivery device, the medicament delivery device having a proximal end pointing towards a dose delivery site during use of the medicament delivery device, and a distal end opposite the proximal end, the medicament delivery device comprising:
a housing comprising a longitudinal axis,
a medicament delivery member cover axially moveable within the housing between an extended position and a retracted position, and
a cap,
wherein the housing comprises a flexible housing arm,
wherein the medicament delivery member cover comprises a blocking recess corresponding to the flexible housing arm,
wherein, when the cap is attached, the cap is configured to bias the flexible housing arm in a radial inward direction, and
wherein the blocking recess is configured to receive the flexible housing arm to block distal movement away from a medicament delivery site of the medicament delivery member cover.

In a non-limiting embodiment, the flexible housing arm is configured to engage with a proximal end surface of the blocking recess.

In a non-limiting embodiment, the flexible housing arm comprises an outer protrusion extending in a radial outward direction.

The invention is specified by the independent claims. Preferred embodiments are defined in the dependent claims. In the following description, although numerous features may be designated as optional, it is nevertheless acknowledged that all features comprised in the independent claims are not to be read as optional.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIGS. 1A and 1B show a medicament delivery member cover according to an example of the present disclosure.

FIGS. 2A and 2B show a cap according to an example of the present disclosure.

FIGS. 3A to 3D show an activation sequence of a medicament delivery device according to an example of the present disclosure.

FIG. 4A shows a medicament delivery member cover according to an example of the present disclosure.

FIG. 4B shows a housing according to an example of the present disclosure.

FIGS. 5A to 5C show an activation sequence of a medicament delivery device according to an example of the present disclosure.

### DETAILED DESCRIPTION

The lockout mechanism according to the present invention can be employed for various medicament delivery devices. Thus, only the parts relevant for carrying out the disclosure will be described in detail.

In the figures, elements not essential for carrying out the invention have been omitted. In particular, not all figures show a complete outer housing, biasing members, a medicament container, cap, etc. in order not to render the depictions overly complicated and not to blur the scope of the disclosure.

In the following, the assembled state refers to a state in which the device is delivered to the patient, i.e., before activation and medicament delivery. In the assembled state, the plunger rod is in a locked position and not able to move in the proximal direction. In the activation state, the medicament delivery member cover is moved in the distal direction along the longitudinal axis from an extended position to a retracted position. In the activated state, the plunger rod is free to move in the proximal direction for medicament delivery. The plunger rod may thus be displaceable along the axial direction.

The medicament delivery member cover can be moved from the extended to the retracted position for activation of the medicament delivery sequence. Therefore, the medicament delivery member cover can be displaced in the axial direction. In the extended position, the medicament delivery member cover extends in a proximal direction from the housing. Thereby, a medicament delivery member, e.g., a needle, may be covered by the medicament delivery member cover. By pushing the medicament delivery member cover in the distal direction, e.g., by pushing the device onto a medicament delivery site, the medicament delivery member cover is moved to the retracted position for activating medicament delivery. After delivery, the medicament delivery member cover may be moved to a final position (locked position) to cover the medicament delivery member. The final position of the medicament delivery member cover may be further proximal than the extended position. The medicament delivery member cover may also be locked in the final position after medicament delivery to prevent injuries of the patient.

The medicament delivery device may comprise a medicament delivery member cover biasing member, for instance a coil spring, configured to exert a force on the medicament delivery member cover 1 in the proximal direction.

In the following, the term engagement encompasses any kind of interaction between the named features such as abutment, clamping, locking, biasing, flexing, etc.

The embodiments described below are examples of the present disclosure and may be combined unless indicated otherwise.

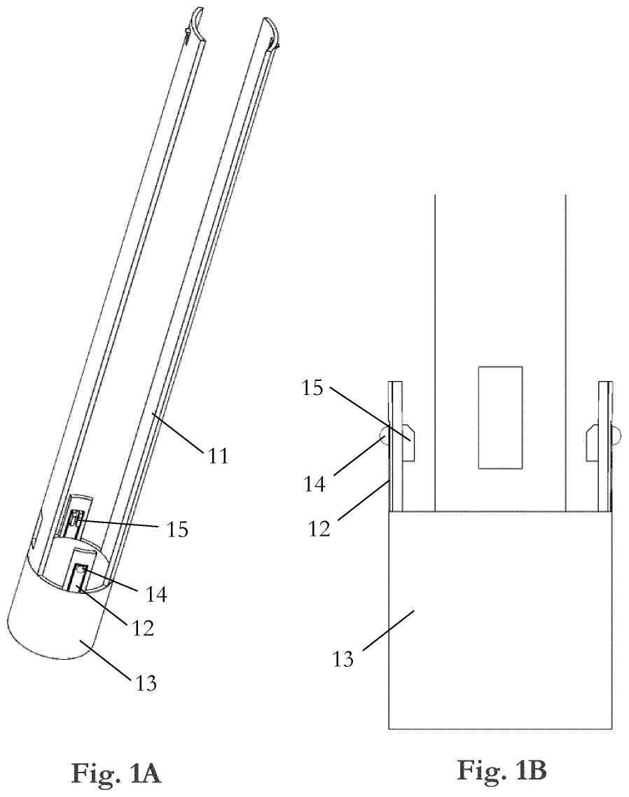

FIG. 1A shows a medicament delivery member cover 1 according to an embodiment. The medicament delivery member cover 1 is axially moveable within a housing 3 between an extended and a retracted position. The medicament delivery member cover 1 comprises a medicament delivery member cover lock 12 being flexible in the radial direction. The medicament delivery member cover 1 may comprise a substantially cylindrical portion 13 at its proximal end and at least one, in particular two, medicament delivery member cover arms 11 extending in the distal direction from the cylindrical portion. The medicament delivery member cover lock 12 may also extend from the cylindrical portion 13. In case there are two medicament delivery member cover locks 12 and two medicament delivery member cover arms 11, they may be offset by 90 degrees with respect to each other in alternating order.

FIG. 1B shows a partial side view of the medicament delivery member cover of FIG. 1A. The medicament delivery member cover lock 12, in the present example two medicament delivery member cover locks 12, comprises an outer protrusion 14. The outer protrusion 14 may extend in the radial outward direction and may be formed as a (spherical) bump, a rib, or similar structure. Hence, the outer protrusion 14 may be sloped at least in the distal direction. The medicament delivery member cover 1 may further comprise an inner protrusion 15 extending in the radial inner direction. The inner protrusion 15 may be formed as a rib, a bump or similar and may also comprise at least one sloped surface.

As shown in FIG. 1A, a protection structure may be formed around the medicament delivery member cover lock 12. The protection structure may consist of a rectangular rib, an arched structure or similar. The medicament delivery member cover lock 12 may be radially flexible within the support structure. In a relaxed state, the medicament delivery member cover lock 12 may substantially extend in the axial direction.

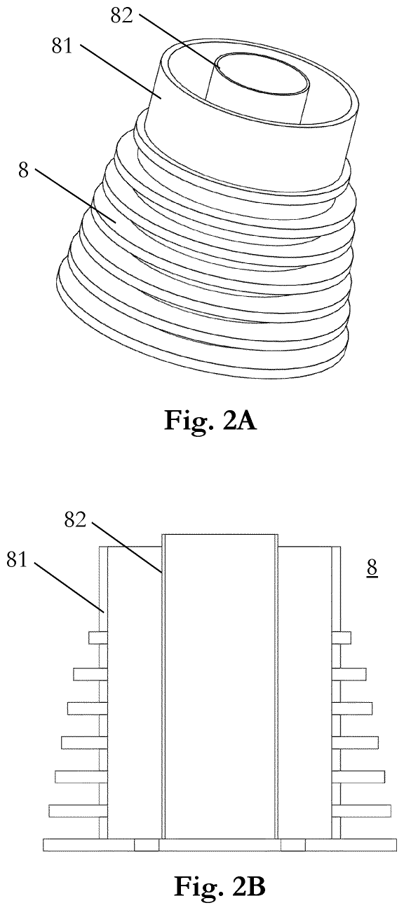

FIG. 2A shows a perspective view of a cap 8 according to an embodiment. FIG. 2B is a sectional view of the cap 8 according to FIG. 2A.

The cap 8 comprises an outer housing 81 and an inner housing 82. The outer housing 81 and the inner housing 82 may be substantially cylindrical and thus also be referred to as tubular members. The inner housing 82 may be provided concentrically inside the outer housing 81.

The space between the outer housing 81 and the inner housing 82 may be configured to receive the proximal end of the medicament delivery member 1.

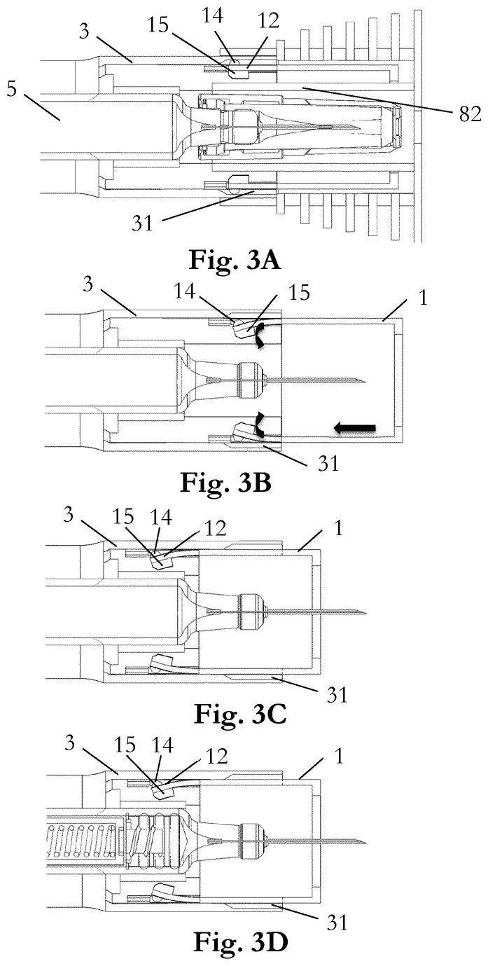

FIGS. 3A to 3D show an activation sequence of a medicament delivery device comprising the elements described above. The figures show sectional views along the longitudinal axis.

FIG. 3A shows a partial view of the medicament delivery device comprising a syringe 5, a housing 3 comprising a longitudinal axis, the cap 8 and the medicament delivery member cover 1, wherein the device is in its initial assembled state as delivered to the patient.

The housing 3 comprises a housing slot 31 configured to receive the outer protrusion 14. The housing slot 31 may be provided at the proximal end of the housing 3. The housing slot 31 may be recessed from the inner circumferential surface of the housing 3 and may extend in the distal (axial) direction.

The housing slot 31 may be sloped at its distal end towards the radial inward direction. Thus, when the medicament delivery member cover 1 is moved in the distal direction to start an injection sequence, the outer protrusion 14 slides along the housing slot 31 and when the distal end of the housing slot 31 is reached, the medicament delivery member cover lock 12 is biased radially inwards due to the interaction of the outer protrusion 14 with the (sloped) end surface of the housing slot 31. However, when the cap 9 is attached to the device and the medicament delivery member cover 1 is moved, e.g., through an impact or vibrations, the medicament delivery member cover lock 12 abuts the inner housing 82, in particular the outer circumferential surface of the inner housing 82, and is thus not free to move in the radial inward direction. An inner protrusion 15 may be provided on the medicament delivery member cover lock 12 to improve the locking mechanism.

Thus, the medicament delivery member cover lock 12 may not be moved in the radial inward direction enough to disengage from the housing slot 31 and distal movement of the medicament delivery member cover 1 may be blocked. In other words, when the medicament delivery member cover 1 is moved distally with the cap 8 attached, the interaction of the outer protrusion 14 with the distal end of the housing slot 31 forces the medicament delivery member cover lock 12 radially inward, but said radial inward movement is blocked by the abutment of the outer protrusion 15 on the inner housing 82 such that the outer protrusion 14 is still engaged with the housing slot 31 and the medicament delivery member cover 1 may not be moved further distally.

With the cap 8 attached, the outer protrusion 14 is configured to abut a distal end of the housing slot 31 when the medicament delivery member cover 1 is moved in the distal direction, thus blocking distal movement of the medicament delivery member cover 1.

An inadvertent activation of the device with the cap 8 attached may thus be prevented.

When the cap 8 has been removed from the device, as shown in FIG. 3B, the medicament delivery member cover lock 12 is free to move radially inward. Thereby, the outer protrusion 14 disengages the housing slot 31 and the medicament delivery member cover 1 is free to be moved in the distal direction to activate the medicament delivery device (see arrows in FIG. 3B).

In FIG. 3C, the medicament delivery member cover 1 has been pushed in the distal direction into the retracted position, e.g., by pushing the medicament delivery member cover onto a medicament delivery site, and the medicament delivery is started. The outer protrusion 14 may slide along the inner circumferential surface of the housing 3.

FIG. 3D shows the device after medicament delivery, i.e., with the plunger rod in the most proximal position.

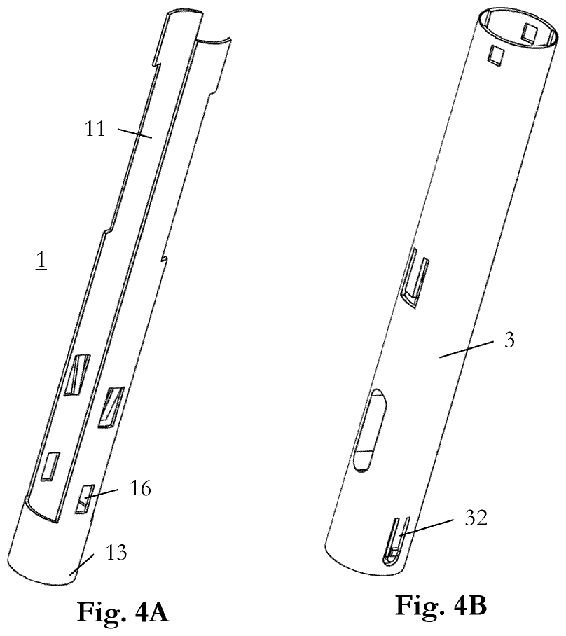

FIG. 4A shows a medicament delivery member cover 1 according to an embodiment. The medicament delivery member cover 1 comprises a blocking recess 16. The medicament delivery member cover 1 may comprise a cylindrical portion 13 and at least one medicament delivery member cover arm 11 extending from the cylindrical portion 13 in the distal direction. The blocking recess 15 may be provided in the medicament delivery member cover arm 11. In particular two medicament delivery member cover arms 11 with a blocking recess 16 each may be provided.

The blocking recess 16 may be formed as a recess, a cavity, or a cut-out on the outer circumferential surface of the medicament delivery member 1.

FIG. 4B is a perspective view of a housing 3 according to an embodiment. The housing 3 comprises a housing arm 32 corresponding to the blocking recess 16. The housing arm 32 may extend in the axial direction and may be flexible in the radial direction. In particular, the housing arm 32 may be able to pivot around its distal end. In other words, the distal end of the housing arm 32 may be connected to the housing 3 and the proximal end may be free to move in the radial direction. The housing arm 32 may further comprise a protrusion such as a bump or a rib extending/protruding in the radial outward direction. The housing arm 32 may be formed as a clip. In the relaxed state, the housing arm 32 substantially extends in the axial direction.

Thus, when the cap 8 is attached to the proximal end of the device, the inner circumferential surface of the cap 8 may interact with the housing arm 32, e.g., the protrusion, to bias the housing arm 32 inward. Thereby, the housing arm 32 engages with the blocking recess 16 of the medicament delivery member cover 1. If the medicament delivery member cover 1 is pushed distally with the cap attached, e.g., through an impact or vibrations, the proximal end surface of the blocking recess 16 abuts the proximal end of the inwardly bent housing arm 32, thus blocking distal movement of the medicament delivery member cover 1 with respect to the housing 3.

The device can therefore not be triggered with the cap attached.

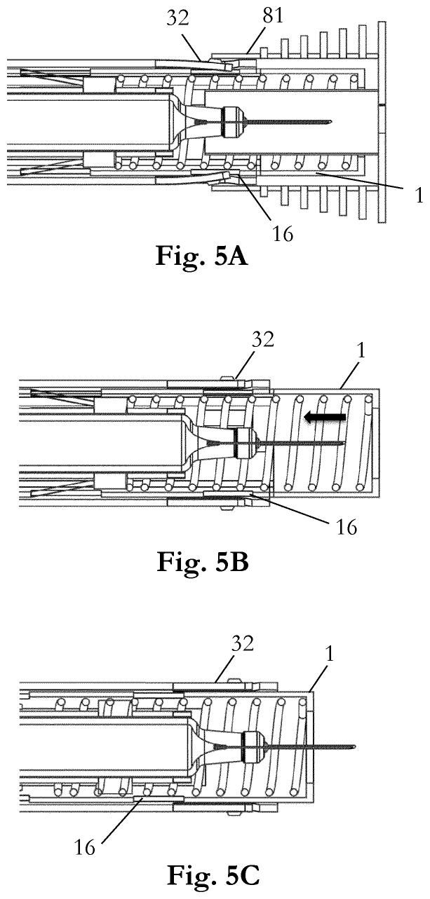

FIGS. 5A to 5C show an activation sequence of a medicament delivery device comprising the elements of FIGS. 4A and 4B.

In FIG. 5A, the cap 8 is attached to the device and the housing arms 32 are flexed inwards due to the interaction of the cap 8 with the protrusion. The blocking recess 16 is configured to receive the housing arms 32. If the medicament delivery member cover 1 is moved distally in that state with the cap 8 attached, the proximal end surface of the blocking recess 16 abuts the inwardly flexed housing arm 32 and further distal movement of the medicament delivery member cover 1 is blocked.

Thus, the flexible housing arm 32 is configured to engage the blocking recess 16 to block distal movement away from a medicament delivery site of the medicament delivery member cover 1.

An inadvertent activation of the device with the cap 8 can thus be effectively prevented.

When the cap is removed, as shown in FIG. 5B, the housing arms 32 are free to flex back into the relaxed state and thus disengage the blocking recess 16.

The medicament delivery member cover 1 can now be moved in the distal direction for activation of the medicament delivery.

FIG. 5C is a depiction of the medicament delivery device in the activated state, i.e., with the medicament delivery member cover 1 in the retracted state.

In the present disclosure, mechanisms for preventing accidentally triggering medicament delivery devices with the cap attached, i.e., safeguard features, have been disclosed. The proposed solutions do not require additional elements but are an effective re-design of already present features and improve the safety of the devices.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5 +2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

Reference Signs

| | |
|---|---|
| 1 | Medicament delivery member cover/needle cover |
| 11 | Medicament delivery member cover arm/needle cover arm |
| 12 | Medicament delivery member cover lock/needle cover lock |
| 13 | Cylindrical portion |
| 14 | Outer protrusion/bump |
| 15 | Inner protrusion/rib |
| 16 | Blocking recess |
| 3 | Housing |
| 31 | Housing slot |
| 32 | Housing arm |
| 5 | Syringe |
| 8 | Cap |
| 81 | Outer housing/outer cap |
| 82 | Inner housing/inner cap/tubular member |

1-8. (canceled)

9. A medicament delivery device having a proximal end pointing towards a dose delivery site during use of the medicament delivery device and a distal end opposite the proximal end, the medicament delivery device comprising:
a housing comprising a longitudinal axis;
a medicament delivery member cover axially moveable within the housing between an extended position and a retracted position; and
a cap,
wherein the cap comprises an outer housing and an inner housing concentrically provided radially inside the outer housing with respect to the longitudinal axis,
wherein a space between the outer housing and the inner housing is configured to receive a proximal end of the medicament delivery member cover extending towards a medicament delivery site,
wherein the medicament delivery member cover comprises a flexible medicament delivery member cover lock being flexible in the radial direction,
wherein the medicament delivery member cover lock comprises an outer protrusion extending in the radial outward direction and being configured to be received in a corresponding housing slot, and
wherein the inner housing is configured to block inward radial movement of the medicament delivery member cover lock.

10. The medicament delivery device according to claim 9, wherein the medicament delivery member cover lock comprises an inner protrusion extending in the radial inward direction.

11. The medicament delivery device according to claim 9, wherein the outer protrusion is configured to abut a distal end of the housing slot when the medicament delivery member cover is moved in the distal direction,
wherein the abutment blocks further distal movement of the medicament delivery member cover.

12. The medicament delivery device according to claim 9, wherein the distal end of the housing slot is sloped radially inwards and configured to bias the medicament delivery member cover lock radially inwards when the medicament delivery member cover is moved in the distal direction.

13. The medicament delivery device according to claim 10, wherein, when the cap is attached to the housing and the medicament delivery member cover is moved in the distal direction, the medicament delivery member cover lock is biased radially inwards and the inner protrusion is configured to abut the inner housing thus blocking radial movement of the medicament delivery member cover lock, and
wherein, when the cap is removed from the housing and the medicament delivery member cover is moved in the distal direction, the medicament delivery member cover lock is biased radially inwards and the outer protrusion is disengaged from the housing slot thus allowing distal movement of the medicament delivery member cover.

14. The medicament delivery device having a proximal end pointing towards a dose delivery site during use of the medicament delivery device and a distal end opposite the proximal end, the medicament delivery device comprising:
a housing comprising a longitudinal axis;
a medicament delivery member cover axially moveable within the housing between an extended position and a retracted position; and
a cap,
wherein the housing comprises a flexible housing arm,
wherein the medicament delivery member cover comprises a blocking recess corresponding to the flexible housing arm,
wherein, when the cap is attached, the cap is configured to bias the flexible housing arm in a radial inward direction, and
wherein the blocking recess is configured to receive the flexible housing arm to block distal movement away from a medicament delivery site of the medicament delivery member cover.

15. The medicament delivery device according to claim 14, wherein the flexible housing arm is configured to engage with a proximal end surface of the blocking recess.

16. The medicament delivery device according to claim 14, wherein the flexible housing arm comprises an outer protrusion extending in a radial outward direction.
